(19) United States
(12) Patent Application Publication
(10) Pub. No.: US 2014/0051652 A1
(43) Pub. Date: Feb. 20, 2014

(54) Treatment for Migraine Headache

(71) Applicant: Kingsley Yianomah QUARTEY, Toronto (CA)

(72) Inventor: Kingsley Yianomah QUARTEY, Toronto (CA)

(21) Appl. No.: 13/590,134

(22) Filed: Aug. 20, 2012

(51) Int. Cl. A61K 31/7048; A61P 25/14; A61P 25/06

(52) U.S. Cl. 514/26

(57) ABSTRACT

Using a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived or Extracted from the Yellow Oleandrin Plant (Oleandrin Peruviana), White Oleandrin Plant (Nerium Oleander) and or other Plants in the Apocynaceae Genus, in the Dogbane Plant Family and or extracted from plants or animals including plants and animals such as Foxglove or synthetic man-made, and or a combination of synthetic and natural thereof in Pharmaceutically Suitable Preparation in a Pharmaceutically Suitable Dose, example orally or intravenously, of which the Prescriber of the said Preparation, or Doctor, based on the overall health of the Patient will prescribe the best Dosage, The Best Mode of Dosage example orally or intravenously, which is best suited to have the Most Desirable Therapeutic Effect

A Pharmaceutically acceptable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleander Plant (Thevetia Peruviana), White Oleander Plant (Nerium Oleander) and or other Plants in the Plantae Kindgdom and Plantae Family of Apocynaceae, Genus in the Dogbane Plant Family and or extracted from plants and or animals including plants and animals such as Foxglove or Synthetic man-made Cardio Glycosides, and or a combination of synthetic and natural Cardio Glycosides such as THEVETIN B, THEVETIN A, PERUVOSIDE, THEVOFOLIN, OLEANDRIN, NERIIFOLIN, THEVETOXIN in Pharmaceutically Suitable Preparation for Treating:
Migraine Headache
Dystonia Dissorder
Traumatic Brain Injury

With the said Pharmaceutically Suitable Preparation being Administered in a Pharmaceutically Acceptable Dosage, in a Pharmaceutically acceptable Mode and or manner, example, such as orally, or intravenously, and etcetera, with the mode and or method and of dosing and dosage depending on the Health and or Physical and Mental condition of the Patient being treated, the Health Care Practitioner and or the Doctor

Prescribing the Said Pharmaceutically Suitable Preparation made with and or Containing Cardio Glycosides for Treating:
Migraine Headache
Dystonia Dissorder
Traumatic Brain Injury

### BACKGROUND OF INVENTION: (i)

### Prior Art

Prior to my Invention, there was no effective Treatment or cure for:
(i) Migraine Headache
(ii) Dystonia Disorder
(iii) Traumatic Brain Injury

That was effective in Treating and or curing (i) Migraine Headache, (ii) Dystonia Disorder and (iii) Traumatic Brain Injury. All this above mentioned diseases and or disorders creates enormous burdens on Families with Individuals that suffers from the above mentioned diseases and or disorders, Societies, and the Sufferers of this debilitating Diseases and or disorders themselves.

### (i) MIGRAINE HEADACHE

Migraine Headache is a chronic disorder that is characterized by a moderate to severe headaches.

Typicaly Migraine Headache affects only One Half of the Head, and symptoms of Migraine Headache includes Extreme Pulsating Headaches, Nausea, Vomiting, Increased Sensitivvity to Light, Touch and Sound.

Migraine Headache is believed to be a Neurovascular Disorder, and there seem to be a substantial link between hyperexcitability of the Celebral Cortex of the Brains of Migraine Headache Sufferers.

It has also been speculated that the phenomenom known as “Corticol Spreading Depression” is the underlying cause of Migraine Headaches.

Recent studies in Migraine Headache has shown that not only do Migraine Headaches cuases sufferers of the ailment severe pains, it (Migraine Heachache) also causes noticeable structural changes in the brain of (Migraine Headache) Sufferes.

While there are medications that are helpful in reducing some of the symptons of Migraine Headache in some Individuals Suffering from it, medications such as for example analgesics “pain killers” such as Aspirin, cannot effectively treat the pulsating and crippling pains from Migraine Headache. This medications do not effectively reduce the pains of Migraine Headache or cure Migraine Headache.

Prior to my Invention there was no Effective Cure or Treatment for Migraine Headache.

(ii) Dystonia Disorder:

Dystonia Disorder is a Neurodegenerative disorder of the central nervous system. It causes characteristic motor problems, such as gait, involuntary shakiness, difficulty in walking and talking, and host of other neurological and psychiatric problems. While it is idiopathic, meaning there is no known cause, it is believed to be caused by genetic and environmental conditions,

Current Treatment:

There are drugs that helps to alleviate some of the symptons in some of the Individuals suffering from Dystonia Disorder, however, there is no cure.

Prior to my Novel Invention which I can demonstrate in any forum if required, there was no known cure for Dystonia Disorder

(iii) Traumatic Brain Injury:

Current Treatment for Traumatic Brain Injury:

Prior to my novel Invention, which I can demonstrate as an effective Treatment for Traumatic Brain Injuryin any forum if required to do so.

Prior to my Invention there was no effective Treatment for Traumatic Brain Injury.

What is claimed is:

1. A Method for Treating Migraine Headache, comprising of a pharmaceutically suitable preparation containing one more Cardio Glycosides such as THEVETIN B, THEVETIN A, PERUVOSIDE, THEVOFOLIN, OLEANDRIN, NERIIFOLIN, and THEVETOXIN;
The one or more Cardio Glycosides could be derived and or extracted from the Yellow Oleandrin Plant (Thevetia Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, and THEVETOXIN thereof in a Pharmaceutically Suitable Preparation such as a Pharmaceutically Suitable “Suspension” that can optionally contain and or include Pharmaceutically suitable Excipients such as Treated Water (Diluent), Propylene Glycol and or Glycerin (Humectants), Carboxyl Methyl cellulose (Suspension Agent and a Polymer), Sodium Benzoate (Preservative) Tocopheryl Acetate (Antioxidant);
The method of dosing could be orally, intravenously, topically, otological drops, nasally, or other pharmaceutically suitable method of dosing, which the Prescriber of the said Preparation, such as a Doctor, based on the overall health of the Patient will prescribe the dosage (unit doses) and the mode of dosing which is will have the most desirable Therapeutic Effect on the patient.
A Method for Treating Dystonia Disorder, comprising of a pharmaceutically suitable preparation containing one more Cardio Glycosides such as THEVETIN B, THEVETIN A, PERUVOSIDE, THEVOFOLIN, OLEANDRIN, NERIIFOLIN, and THEVETOXIN;
The one or more Cardio Glycosides could optionally be derived and or extracted from the Yellow Oleandrin Plant (Thevetia Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, and THEVETOXIN thereof in a Pharmaceutically Suitable Preparation such as a Pharmaceutically Suitable “Suspension” that can optionally contain and or include Pharmaceutically suitable Excipients such as Treated Water (Diluent), Propylene Glycol and or Glycerin (Humectants), Carboxyl Methyl Cellulose (Suspension Agent and a Polymer), Sodium Benzoate (Preservative) Tocopheryl Acetate (Antioxidant);
The method of dosing could be orally, intravenously, topically, otological drops, nasally, or other pharmaceutically suitable method of dosage (unit doses),
which the Prescriber of the said Preparation, such as a Doctor, based on the overall health of the Patient will prescribe the dosage (unit doses) and the mode of dosing which will have the most desirable Therapeutic Effect on the patient.
A Method for Treating Headache, comprising of a pharmaceutically suitable preparation containing one more Cardio Glycosides such as THEVETIN B, THEVETIN A, PERUVOSIDE, THEVOFOLIN, OLEANDRIN, NERIIFOLIN, and THEVETOXIN; The one or more Cardio Glycosides could be derived and or extracted from the Yellow Oleandrin Plant (Thevetia Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, and THEVETOXIN thereof in a Pharmaceutically Suitable Preparation such as a Pharmaceutically Suitable “Suspension” that can optionally contain and or include Pharmaceutically suitable Excipients such as Treated Water (Diluent), Propylene Glycol and or Glycerin (Humectants), Carboxyl Methyl Cellulose (Suspension Agent and a Polymer), Sodium Benzoate (Preservative) Tocopheryl Acetate (Antioxidant);
The method of dosing could be orally, intravenously, topically, otological drops, nasally, or other pharmaceutically suitable method of dosage (unit doses), which the Prescriber of the said Preparation, such as a Doctor, based on the overall health of the Patient will prescribe the dosage (unit doses), the mode of dosing which is will have the most desirable Therapeutic Effect on the patient.
